(19) United States
(12) Patent Application Publication
(10) Pub. No.: US 2005/0033448 A1
(43) Pub. Date: Feb. 10, 2005

(54) Body implantation tube

(76) Inventor: Hoon-Bum Lee, Kangnam-gu, Seoul (KR)

Correspondence Address: Ladas & Parry, 5670 Wilshire Boulevard, Suite 2100, Los Angeles, CA, 90036-5679, US

(21) Appl. No.: 10/479,288

(22) PCT Filed: Jun. 7, 2002
(86) PCT No.: PCT/KR02/01077

(30) Foreign Application Priority Data
Jun. 7, 2001 (KR) 2001/31782

(51) Int. Cl. A61F 2/04

(52) U.S. Cl. 623/23.67; 623/23.68; 623/1.36

(57) ABSTRACT

The present invention relates to a body implantation tube to be used for normalizing the flow of fluid in tubular organs of human body, injured by disease or accident, and comprising, a tubular body member with a longitudinal passage; one or more expanding-fixing members surrounding the body member, the expanding-fixing member being expanded, by absorbing the fluid flowing along the passage of the body member, to be fixed, in a tubular organ as a result of expansion; and a plurality of holes connecting the longitudinal passage of the body member to the expanding-fixing member, the holes being formed on the portion of the body member contacting to the expanding-fixing member.

### TECHNICAL FIELD

The present invention relates to a body implantation tube to be used for normalizing the flow of fluid in tubular organs of human body, injured by disease or accident, and in particular, the implantation tube which is inserted in the tubular organs of human body, in which the flow of fluid takes place, such as lacrimal tube, and then is expanded by the water component in the fluid and fixed, thereby substituting the injured region to allow the fluid to flow smoothly until the injured region regenerates or permanently.

### BACKGROUND ART

Human body is an organization of continuing the life action in which liquid phase materials such as bloods, body fluids, secreting fluids, excretions, etc. flow continuously. There are many tubular organs, as passages for movement of these liquid phase materials, in the human body: for example, lacrimal tube, urethral canal, biliary tract, etc. When one or more these tubular organs are clogged by a disease or accident, the liquid phase material cannot be circulated, whereby a patient suffers pain and may be attacked with other diseases. For example, when the lacrimal tube is clogged, tears, being a secreting fluid secreted toward the eyes, cannot flow inside the nasal cavity and instead flows along the surface of face, whereby the nasal cavity becomes dry. It has been reported that the obstruction of lacrimal tube is a very general phenomenon which occurs in one or two peoples per one hundred of traffic accident patients. Meanwhile, when the urethral canal is clogged, the urine filtered in the kidney cannot move toward the urinary bladder so that the kidney tissue may be destroyed, whereby a patient may lose his life ultimately.

Such occlusion phenomenon of tubular organs is generally treated by a physical operation, and assistant devices are generally used to penetrate the clogged site. For example, as methods of treating the clogged lacrimal tube, there are the method of inserting a funnel-shaped silicone tube (so called, “Olive tube”) or a glass tube (so called, “Jones tube”) into the clogged lacrimal tube, and the method of inserting the silicone tube of an open loop shape into the lacrimal tube and then binding both ends of loop inside or outside the nasal cavity. These tubes remain in the insertion state for a certain period (generally three weeks, in any case six weeks) and then are removed when the injured site recovers. However, in the Olive tube, the funnel-shaped end which is fixed on the entrance of lacrimal tube disturbs the blinking of eye to suffer a patient and also tends to fall out in sneezing or other external imparts. In the silicone tube of the loop shape, the part of the silicone tube is externally exposed, thereby having a bad effect on a patient's appearance or act. Also, external materials or germs may enter the human body along the loop due to the movement of loop.

In order to solve these problems, U.S. Pat. No. 3,726,284 provides a lacrimal drainage duck having an expanded portion, being made of glass or rigid plastic, on the center of a cylindrical tube. However, it is difficult to insert this duck into the lacrimal tube because the expanded portion must have a large dimension for its fixation in the lacrimal tube so that the injured region needs to be incised to the fair extent for its insertion. U.S. Pat. No. 5,318,513 provides a fixation stent including a flexible tube having a proximal end, a distal end, an inflatable portion in fluid communication with the distal end, and a plug for sealing and anchoring the proximal end within a body canal. However, a portion of the stent is exposed to the eye, thereby disturbing the blinking of eye.

Meanwhile, as a method of treating the clogged urethral canal, there is the method of using a long, cylindrical tube, in which both ends of the tube are rolled like the pig tail and a wire is inserted into a longitudinal passage of the tube. In operation, the tube is inserted into the urethral canal, and when the wire is removed from the tube, the both ends are rolled to their original shape, with one of end hanging on the entrance of the kidney and the other end hanging on the entrance of the urinary bladder, so that the tube passes the urethral canal. However, germs in the urinary bladder may enter the kidney along the tube and pollute it. Moreover, the tube inhibits or damages the action of sphincter around the urinary bladder and, as a result, the urine may flow continuously in the urinary bladder.

As a device of expanding the blood vessel where the occlusion phenomenon takes place, a tubular unit is disclosed in U.S. Pat. No. 3,889,684, which comprises a tube having an open end and a closed end, a compressible cuff surrounding the tube and mounted on the tube adjacent the closed end thereof. This tubular unit works as expanding the blood vessel where the occlusion phenomenon takes place now, and simultaneously removing the thrombus accumulated therein, by inserting the unit into the blood vessel beyond the occluded region thereof, and injecting water through the open end to expand the cuff, and then drawing the unit out the vessel. However, the expanded region of the blood vessel may shrink after the operation, and the interior surface of vessel is damaged so that new tissues may grow thereon. Furthermore, this tubular unit cannot be used in other tubular organs such as lacrimal tube, urethral canal, etc.

### SUMMARY OF INVENTION

The objects of the present invention are to solve the problems described above for once and all.

An object of the present invention is to provide a novel body implantation tube of being inserted in the injured region of human body in an endoscopic operation and then being automatically fixed therein to make the flow of body fluids, secreting fluids, excretions, etc. smooth.

Another object of the present invention is to provide a novel body implantation tube of being impanted in the human body to substitute a tubular organ, the tubular organ being unable to regenerate because of the serious injury thereof.

A still further object of the present invention is to provide a novel body implantation tube of blocking the inflow of external materials to accelerate the regeneration rate of a tubular organ, the tubular organ having a long length and a large injured region.

In order to accomplish these objects, the body implantation tube of the present invention comprises,
a tubular body member with a longitudinal passage;
one or more expanding-fixing members surrounding the body member, the expanding-fixing member being expanded, by absorbing the fluid flowing along the passage of the body member, to be fixed in a tubular organ as a result of expansion; and
a plurality of holes connecting the longitudinal passage of the body member to the expanding-fixing member, the holes being formed on the portion of the body member contacting to the expanding-fixing member.

The length of tubular body member may change depending on the length of a tubular organ on which the tube of the present invention will be implanted. One of the features of the present invention is to be able to treat an injured region just by installing simply the implanting tube on the injured region of the tubular organ, so that the implanting tube needs not to extend from the entrance of the tubular organ to its exit. For example, when the middle region of the lacrimal tube is injured, the implanting tube of the present invention needs not to be exposed at the entrance of the lacrimal tube, i.e., a region adjacent to the eye, unlike methods of the prior art, thereby reducing a patient's pain. When the middle region of urethral canal is injured, the implanting tube of the present invention needs not to extend to the entrance of urethral canal and the exit of urethral canal, in which the entrance means a region adjacent to the kidney and the exit means a region adjacent to the urinary bladder, thereby not damaging the action of sphincter.

The tubular body member may be of a mere cylindrical shape, or be of a funnel shape in which one or both ends of the body member have a relatively larger diameter than a middle portion thereof. Especially, the funnel-shaped body member is available to gather the secretion such as tears through a funnel-shaped end (“proximal end”) and to send it toward the opposite end (“distal end”). In another embodiment, in order to facilitate insertion into the tubular organ, the plane of the distal end is not vertical but inclined. The outer surface of this end is preferably round so as to not damage the interior surface of the tubular organ in insertion.

The diameter of the tubular body member is not particular limited. One of the features of the present invention is to be able to guide the flow of fluid, without having a reverse effect on the action of tubular organ, even in the case that the outer diameter of the body member is smaller than the inner diameter of the tubular organ. In the tubular organs of the human body, such as urinary canal, the peristalsis occurs, i.e., a series of normal coordinated, rhythmic muscle contractions that occurs automatically to move food through the digestive tract, urine from the kidneys through the ureters into the bladder, and bile from the gallbladder into the duodenum. When the outer diameter of implantation tube is the same or a little smaller than the inner diameter of tubular organ, as in the conventional implantation tube, the peristalsis is inhibited and thus the smooth flow of fluid cannot be induced. However, in the implantation tube of the present invention, the fluid can also flow through the expanding-fixing member so that, as a result, the fluid can flow on the interior and exterior of the tubular body member, respectively. Therefore, even in the case that the outer diameter of body member is further smaller than the inner diameter of tubular organ, the body member can be fixed to the interior of tubular organ by the expanding-fixing member, thereby guiding the flow of fluid without disturbing the peristalsis of tubular organ.

The expanding-fixing member has a little larger diameter than the outer diameter than of body member in the state of not absorbing a fluid, but expands to about three-fold to twenty-fold extent in the state of absorbing a fluid. In consideration that the expanding-fixing member expands, with the implantation tube inserted in the tubular organ, the expansibility is remarkably restricted by the tubular organ, but the implantation tube can be fixed in the tubular organ due to such expansion. Body fluids, secreting fluids and the like in vivo contain the quite amount of water component, and the water component flows through a plurality of holes perforated on the body member to swell the expanding-fixing member.

The shape of the expanding-fixing member is not particularly limited, but preferably, the overall shape is a cylinder being concentric to the tubular body member and its both ends are inclined so as to contact gently to the body member.

The joining between the expanding-fixing member and the body member can be achieved in various ways; attaching the expanding-fixing member to the body member with an adhesive, making the outer surface of body member irregular and then molding the expanding-fixing member thereon, etc. In the attaching way, the biocompatible adhesive should be used because the components of adhesive may bleed from the joining portion in expansion of the expanding-fixing member. As a commercial adhesive available, is there Silastic® (DowCorning Corporation). In the molding way, the body member with the irregular surface is first molded and then the expanding-fixing member is incorporated therewith by molding.

Because the implantation tube of the present invention is a device to be inserted in the human body, it should be biocompatible. Many materials with the biocompatibility have been known in the art: for the body member, styrene/ethylene/butylenes copolymer (C-Flex®), polyethylene copolymer (Percuflex®), polyester copolymer (Silitek®), etc. can be used but not limited to them. Silicone of them is particularly desirable because of its good resilience, easy moldability and low price.

The expanding-fixing member should be expanded in absorption of water, as well as biocompatibility. Preferable examples of them include porous polyvinyl alcohol, and porous polymers obtained by partially crosslinking polyvinyl alcohol with formaldehyde, acetyl aldehyde and the like. The commercial material available is Merocel® (Merocel Corporation) of which the detailed information is disclosed in U.S. Pat. No. 4,098,728, which is incorporated with the present invention as a reference.

In a embodiment, the expanding-fixing member is further coated with a material with the good elasticity and biocompatibility or covered a thin sheet to prevent the expanding-fixing member from contacting directly to in vivo tissues and restrict the expansion thereof to the appropriate extent and prevent the calcification thereof caused by contacting to calcium. Such configuration protects the interior wall of tubular organ from the implantation tube efficiently.

In another embodiment, a plurality of holes are not formed between the tubular body member and expanding-fixing member and instead a water-supplying means is further included, the water-supplying means supplying water to the expanding-fixing member and then being able to be removed.

The implantation tube of this configuration comprises, a tubular body member with a longitudinal passage; one or more expanding-fixing members surrounding the body member, the expanding-fixing member being expanded, by absorbing the fluid flowing along the passage of the body member, to be fixed in a tubular organ as a result of expansion; and a water-supplying means supplying water to the expanding-fixing member, the water-supplying means being joined to the expanding-fixing member and being able to be removed therefrom after the supply of water. The water-supplying means is positioned inside or outside the longitudinal passage of the body member. The joining portion between the expanding-fixing member and the water-supplying means is configured to be readily divided in applying a pulling force thereto.

In another embodiment, an implantation tube of the present invention comprises a first implantation tube (A) of the relatively small dimension and a second implantation tube (B) of the relatively large dimension, wherein the outer diameter of the first implantation tube (A) is smaller than the inner diameter of the second implantation tube (B) so that, in the state that the second implantation tube (B) is fixed in the human body, the first implantation tube (A) is inserted and fixed in the longitudinal passage of the second implantation tube (B). While the second implantation tube (B) is almost identical with the implantation tube as illustrated earlier, the first implantation tube (A) may have a longitudinal passage as in the second implantation tube (B) or may not so.

The implantation tube combination of this configuration is useful to control the amount of fluid which flows through the longitudinal passage of the second implantation tube (B) having been already fixed in the human body.

The implantation tubes of the present invention are used to treat the tubular organs in the human body, such as, in addition to lacrimal tube and urethral canal as mentioned earlier, nasolacrimal duct, ductus pancreaticus, ureter, parotid duct, fallopian tube, deferent duct, etc. but are not limited them.

When a tubular organ has regenerated with the aid of an implantation tube and needs to be removed, it can be removed by applying a biocompatible lubricant on the outer surface of expanding-fixing member and then pulling the tube.

As shown below, the description refers to the drawing in order to describe the present invention more in detail, thereby, the scope of the invention is however not to be interpreted as a limitation of the invention.

### DETAILED DESCRIPTION OF PREFERRED EMBODIMENTS

FIG. 1 shows an implantation tube according to an embodiment of the present invention. The implantation tube 100 is of a hollow, long cylinder and acts like a tubular organ in the human body. The proximal end 112 of the implantation tube 100 is configured to be of a funnel shape to have a larger diameter than a tubular body member 110. This configuration is useful for gathering, for example, tears through the proximal end 112. A distal end 114 is inclined to be easily inserted into a tubular organ (not shown). Accordingly, the implantation tube 100 is inserted into the tubular organ starting the distal end side 114. An expanding-fixing member 200 surrounds a part of the tubular body member 110, wherein the expanding-fixing member 200 in operation will be positioned, for example, on the lacrimal sec with a relatively small inner diameter in the lacrimal tube. The proximal end 202 and distal end 204 of the expanding-fixing member 200 is inclined toward the tubular body member 110, thereby having no significant effect on the interior wall of the tubular organ in insertion of the implantation tube 100.

FIG. 2A shows an axial cross-sectional view of the implantation tube 100 of FIG. 1. A plurality of holes 120, working as connecting the interior space of a tubular body member 110 to an expanding-fixing member 200, are formed on the portion of the body member 110 to which the expanding-fixing member 200 joins. There are various ways of joining the expanding-fixing member 200 to the body member 110. In addition to a way of using an adhesive and a way of making the surface of body member irregular and then molding the expanding-fixing member thereon, as illustrated earlier, another possible way is to mold a polymer-raw material for the expanding-fixing member 200 directly on the tubular body member 110 on which the holes 120 are formed. Herein, a part of polymer, being of a liquid phase or melting phase, flows into the holes 120, thereby achieving the joining with the body member 110.

FIG. 2B shows an axial cross-sectional view of an implantation tube 100 which was swelled by the fluid flowing through the longitudinal passage of the tubular body member 110. A part of the fluid, flowing through the longitudinal passage of body member 110, flows in an expanding-fixing member 200 through holes 120, and the influx fluid is absorbed in the expanding-fixing member 200. As the expanding-fixing member 200 is swelled by the water component of fluid, the expanding-fixing member expands. In any case, the expansion may be caused by the water component, being in the interior wall of a tubular organ on which the implantation tube 100 will be implanted. Although the expansion is depicted to take place vertically in the figure, the expanding-fixing member 200 expands along the circumferential surface of the body member 110 to form a donut shape. The expanding-fixing member 200 expanded thus contacts to the interior wall of the tubular organ (not shown), thereby fixing the implantation tube 100 at the specific position. Even in the case that the outer diameter of the body member 110 is further smaller than the inner diameter of the tubular organ, the implantation tube 100 can be fixed by the expansion of expanding-fixing member 200. Accordingly, it is possible to insert the implantation tube 100 through a small incision in an endoscopic operation, without incising directly the injured tubular organ or opening a large region adjacent thereto. The inserted tube 100 is first swelled by the water component being in the interior wall of tubular organ and then further swelled by the water component flowing through the holes 120 of tubular body member 110. The expansion of implantation tube 100 is considerably restricted by the interior wall of tubular organ, thus, the expansion progresses horizontally along the tubular body member 110.

FIG. 3 shows an implantation tube 300 of another configuration. A thin film 210 covers the outer surface of an expanding-fixing member 200. As mentioned earlier, the thin film 210 acts as preventing the expanding-fixing member 200 from contacting directly to the tissue of tubular organ, limiting the expansibility to the appropriate extent, and preventing the calcification to be caused by the contact of calcium.

FIG. 4A shows an implantation tube 400 of another configuration. The implantation tube 400 does not include a plurality of holes 120, unlike the implantation tube 100 of FIG. 2A. Instead, a small water-supplying tube 500, which works as supplying water for swelling of an expanding-fixing member 200, is connected to the expanding-fixing member 200. Even though the water-supplying tube 500 is positioned inside a tubular body member 110 in the figure, it may also be positioned outside. In the configuration of FIG. 4A, one hole 130, to which the end 510 of water-supplying tube 500 joins, is perforated on the body member 510. The joining between the end 500 and the hole 130 can be disassembled by pulling the water-supplying tube 500. The hole 130 can be of various configurations; in another embodiment of FIG. 4B, the hole 130 is configured to prevent the water component of the expanding-fixing member 200 from being discharged. Referring to FIG. 4B, a covering means 132 is joined to a tubular body member 110 by a hinge 134, and a sealing means 136 is attached to the corresponding portion of the body member 110. Therefore, as the pressure takes place by flow of a fluid in the tubular body member 110, the covering means 132 pivots on the hinge 134 in the arrow direction to close the hole 130, whereby the fluid flowing inside the body member 110 cannot be discharged toward the expanding-fixing member 200. Configurations for preventing the discharge of fluid are not limited to the configuration of FIG. 4B.

FIG. 5 shows an implantation tube 600 of another configuration which includes two expanding-fixing members. The implantation tube 600 is made of a biocompatible and elastic silicone so that the tube 600 can easily bend. Two expanding-fixing members 310, 320 are of the same configuration with the expanding-fixing member 200 of FIG. 3 which is covered with a thin protecting film, and are installed near both ends 612, 614 of a tubular body member 610. The implantation tube 600 of such configuration is useful for treatment of a tubular organ having a long length and large injured region. Especially, when the injured region of tubular organ is placed between two expanding-fixing members 310, 320, the influx of extraneous substance is blocked by the expanding-fixing members 310, 320, whereby the injured region is protected and the regeneration rate can be accelerated.

FIG. 6 shows another implantation tube of the present invention. The feature of the implantation tube of FIG. 6 is the combination of two tube elements of different configurations.

Referring to FIG. 6, a first implantation tube 700 of a relatively small dimension is inserted into a second implantation tube 800 of a relatively large dimension. The inner diameter of the second implantation tube 800, forming the exterior in the state of combination, is larger than the outer diameter of the first implantation tube 700. Such combination is useful in the case that an implantation tube should be semipermanently implanted in the human body because of serious damage of a tubular organ, in the case that it take long time for an injured tubular organ to regenerate, and in the case that the flowing amount of fluid needs to be controlled in the state the second implantation tube has been implanted for a long time. While the second implantation tube 800 is fixed to a tubular organ, various first implantation tubes 700 with different inner diameters can be alternatively inserted thereto at need. Since the control of flowing amount includes the case of complete blocking, a tubular body member 710 may be of the configuration that the longitudinal passage is sealed. Such modifications should be interpreted to be included in the scope of the present invention.

In order for the first implantation tube 700 to be easily inserted into the second implantation tube 800 and seal completely the open proximal end 820 of a tubular body member 810 of the second implantation tube 800, as in FIG. 6, the proximal end 712 of a tubular body member 710 of the first implantation tube 700 is of a funnel shape, and the outer diameter of the proximal end 712 is the same or larger than the inner diameter of the proximal end 820. In another embodiment, the proximal end 712 of the tubular body member 710 is not of a funnel shape but of the mere cylinder shape. In this case, the outer diameter of the first implantation tube 700 is smaller than the inner diameter of the second implantation tube 800.

The present invention being thus described, it will be obvious that the same may be varied in many ways. Such variations are not to be regarded as a departure from the spirit and scope of the invention and all such modifications would be obvious to one skilled in the art.

### BRIEF DESCRIPTION OF THE DRAWINGS

FIG. 1 is a perspective view of an implantation tube according to an embodiment of the present invention.

FIG. 2A is an axial cross-sectional view of the implantation tube of FIG. 1.

FIG. 2B is an axial cross-sectional view of the implantation tube of FIG. 1, which was swelled by influx of a fluid being in the interior of implantation tube.

FIG. 3 is a perspective view of an implantation tube according to another embodiment of the present invention.

FIGS. 4A and 4B are a perspective view of an implantation tube according to a further embodiment of the present invention, and a partially magnified view of a hole.

FIG. 5 is an axial cross-sectional view of an implantation tube according to a still further embodiment of the present invention, which includes two expanding-fixing members.

FIG. 6 is a perspective view of the combination of two implantation tubes of different dimensions in the state of using.

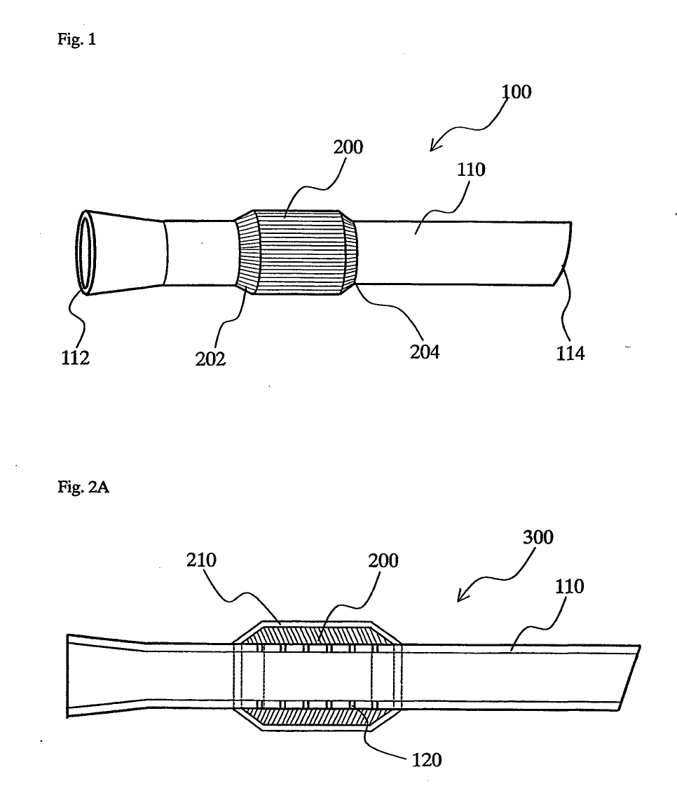

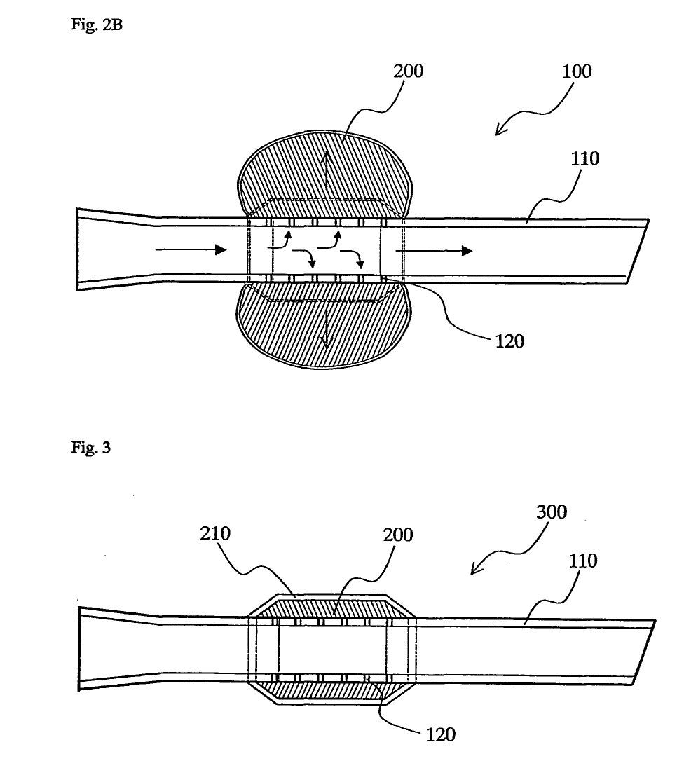

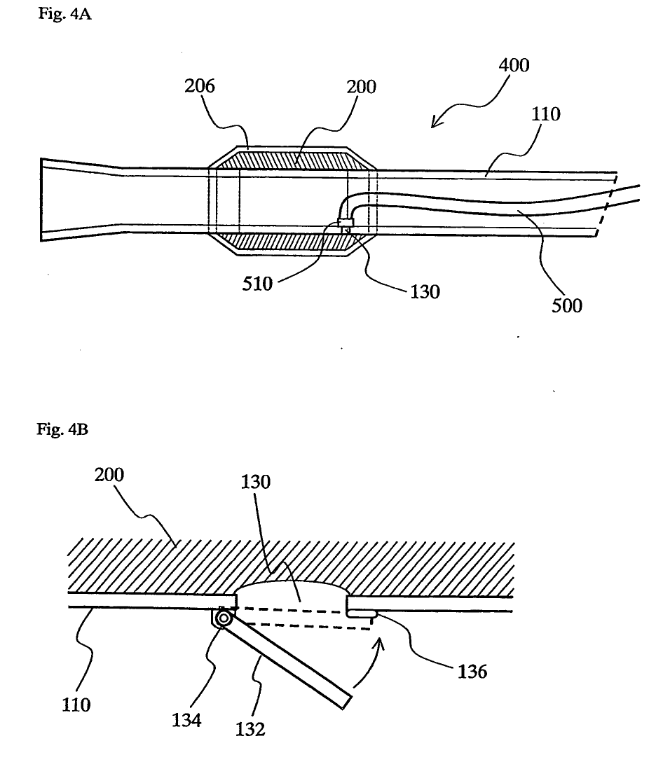

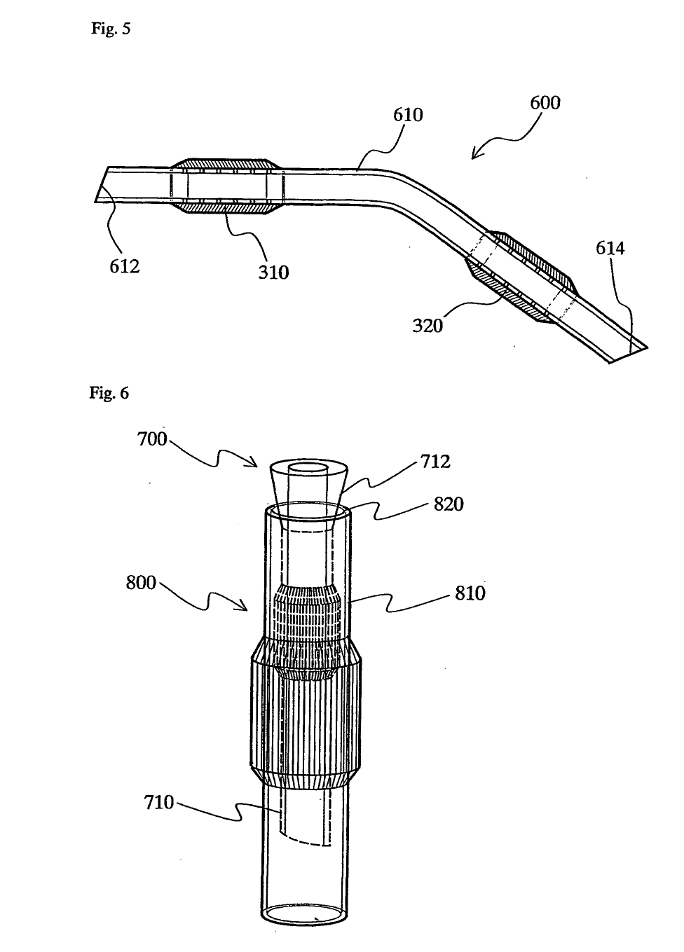

### DESIGNATION OF THE REFERENCE NUMBERS

100, 300, 400, 600, 700, 800: body implantation tube
110, 610, 710, 810: tubular body member
120: holes
200, 310, 320: expanding-fixing member

### INDUSTRIAL APPLICABILITY

The body implantation tube of the present invention is readily inserted into a tubular organ of the human body and then automatically fixed, thereby reducing the operative time remarkably. In the case of lacrimal tube, the implantation tube does not extend to the entrance of tubular organ, i.e., toward the eye, thereby having no adverse effect on the eyes and the appearance of a patient. In the case of urethral canal, the implantation tube can be used to form a new urinary tract only in an injured region thereof without any auxiliary element to hang the kidney and urinary bladder, thereby not causing nephritis, cystitis, injury of sphincter, etc. Furthermore, the implantation of tube can be performed in an endoscopic operation.

1. A body implantation tube comprising,
a tubular body member with a longitudinal passage;
one or more expanding-fixing members surrounding the body member, the expanding-fixing member being expanded, by absorbing the fluid flowing along the passage of the body member, to be fixed in a tubular organ as a result of expansion; and
a plurality of holes connecting the longitudinal passage of the body member to the expanding-fixing member, the holes being formed on the portion of the body member contacting to the expanding-fixing member.

2. The body implantation tube according to claim 1, wherein the tubular body member is of a mere cylindrical shape, or a funnel shape in which one or both ends of the body member have a relatively larger diameter than a middle portion thereof.

3. The body implantation tube according to claim 1, wherein the plane of the distal end is not vertical but inclined to be easily inserted into the tubular organ.

4. The body implantation tube according to claim 1, wherein the expanding-fixing member is further coated with a material with the good elasticity and biocompatibility or covered a thin sheet to prevent the expanding-fixing member from contacting directly to in vivo tissues and restrict the expansion thereof to the appropriate extent and prevent the calcification thereof caused by contacting to calcium.

5. The body implantation tube according to claim 1, wherein the tubular organ, into which the body implantation tube is inserted, is lacrimal tube, urethral canal, nasolacrimal duct, ductus pancreaticus, ureter, parotid duct, fallopian tube, deferent duct, etc.

6. A body implantation tube comprise,
a tubular body member with a longitudinal passage;
one or more expanding-fixing members surrounding the body member, the expanding-fixing member being expanded, by absorbing the fluid flowing along the passage of the body member, to be fixed in a tubular organ as a result of expansion; and
a water-supplying means supplying water to the expanding-fixing member, the water-supplying means being joined to the expanding-fixing member and being able to be removed therefrom after the supply of water.

7. A body implantation tube comprising a first implantation tube (A) of the relatively small dimension and a second implantation tube (B) of the relatively large dimension, the first and second implantation tube (A), (B) being almost identical with the implantation tube of claim 1, the first implantation tube (A) having a longitudinal passage or not so,
wherein the outer diameter of the first implantation tube (A) is smaller than the inner diameter of the second implantation tube (B) so that, in the state that the second implantation tube (B) is fixed in the human body, the first implantation tube (A) is inserted and fixed in the longitudinal passage of the second implantation tube (B).
